(19) United States
(12) Patent Application Publication
(10) Pub. No.: US 2010/0172984 A1
(43) Pub. Date: Jul. 8, 2010

(54) TABLET DOSAGE FORM COMPRISING CETIRIZINE AND PSEUDOEPHEDRINE

(76) Inventors: Bijay Kumar Padhi, Ganjam (IN); Anurag Sood, Kullu (IN); Ashok Kumar Rampal, Amritsar (IN)

Correspondence Address: Ranbaxy Inc., Intellectual Property Department, 600 College Road East, PRINCETON, NJ, 08540, US

(21) Appl. No.: 12/303,609

(22) PCT Filed: Jun. 6, 2007
(86) PCT No.: PCT/IB07/52136

§ 371 Date: Apr. 21, 2009

(30) Foreign Application Priority Data
Jun. 6, 2006 (IN) 1356/DEL/2006

(51) Int. Cl. A61K 9/24; A61K 9/22; A61K 31/495; A61P 37/08

(52) U.S. Cl. 424/472; 424/468; 514/255.04

(57) ABSTRACT

The present invention relates to a tablet dosage form comprising an immediate release component comprising cetirizine and an extended release component comprising pseudoephedrine.

### FIELD OF THE INVENTION

The present invention relates to a tablet dosage form comprising an immediate release component comprising cetirizine and an extended release component comprising pseudoephedrine.

### BACKGROUND OF THE INVENTION

Cetirizine is a generic name for 2-[2-[4-[(4-chlorophenyl)phenylmethyl]-1-piperazinyl]ethoxy]-acetic acid and is typically provided as a dihydrochloride salt. Cetirizine is an orally active and selective H1-receptor antagonist currently prescribed for the treatment of seasonal allergies in patients aged 2 years and older.

Pseudoephedrine, as well as pharmaceutically acceptable acid addition salts thereof such as the hydrochloride and sulfate salts, is a sympathomimetic drug known by those skilled in the art as a safe therapeutic agent for treating nasal congestion. It is commonly administered orally and concomitantly with an antihistamine for treatment of nasal congestion for the treatment of allergic rhinitis.

U.S. Pat. No. 6,469,009 discloses the combined use of pseudoephedrine, an individual optical isomer or a pharmaceutically acceptable salt thereof and cetirizine, an individual optical isomer or a pharmaceutically acceptable salt thereof in the treatment of rhinitis, cold, flu, cold-like and flu-like symptoms.

U.S. Pat. No. 6,171,618 discloses a solid dosage form comprising cetirizine and pseudoephedrine wherein at least a portion of said pseudoephedrine is contained in a core, whereby release of said pseudoephedrine into an environment of use is sustained; wherein said cetirizine is contained as an immediate-release component in said dosage form; and wherein said dosage form is substantially free of alcohols having a molecular weight lower than 100 and reactive derivatives thereof. It further states that contact of cetirizine with alcohols having a molecular weight less than 100 should be avoided since such contact can result in a reaction with cetirizine, usually esterification, and thereby damage the dosage form by reacting with the active medicinal agent.

U.S. Pat. No. 7,014,867 discloses a tablet comprising at least two distinct segments, one segment of which comprises as active ingredient predominantly cetirizine and a second segment of which comprises as active ingredient predominantly pseudoephedrine, said segments being composed and formed in such a way that the resulting tablet is substantially free of impurities formed by reaction of cetirizine with pseudoephedrine, wherein the interfacial surface area of the pseudoephedrine segment and cetirizine segment is less than 180 mm².

Applicants have now developed an alternative tablet dosage form comprising an immediate release component comprising cetirizine or an optically active isomer thereof or pharmaceutically acceptable salt thereof and an extended release component comprising pseudoephedrine or pharmaceutically acceptable salts thereof, wherein interfacial surface area between cetirizine and pseudoephedrine component is more than 180 mm². The tablet dosage forms obtained according to the present invention were found to be stable during the entire shelf life of the product.

Hence, according to one of the aspects, there is provided a tablet dosage form comprising
a) an immediate release component comprising cetirizine or an optically active isomer thereof or pharmaceutically acceptable salt thereof, and
b) an extended release component comprising pseudoephedrine or pharmaceutically acceptable salts thereof,
   wherein the interfacial surface area between cetirizine and pseudoephedrine component is more than 180 mm².

According to another aspect, there is provided a process for the preparation of a tablet dosage form comprising
a) an immediate release component comprising cetirizine or an optically active isomer thereof or pharmaceutically acceptable salt thereof, and
b) an extended release component comprising pseudoephedrine or pharmaceutically acceptable salts thereof,
   wherein the interfacial surface area between cetirizine and pseudoephedrine component is more than 180 mm².

According to another aspect, there is provided a method for treating a disorder selected from rhinitis, cold, flu, cold-like and flu-like symptoms in a human comprising administering a tablet dosage form comprising
a) an immediate release component comprising cetirizine or an optically active isomer thereof or pharmaceutically acceptable salt thereof, and
b) an extended release component comprising pseudoephedrine or pharmaceutically acceptable salts thereof,
   wherein the interfacial surface area between cetirizine and pseudoephedrine component is more than 180 mm².

According to one of the aspects, there is provided a tablet dosage form comprising
a) an immediate release component comprising cetirizine or an optically active isomer thereof or pharmaceutically acceptable salt thereof,
b) an extended release component comprising pseudoephedrine or pharmaceutically acceptable salts thereof, and
c) alcohols having a molecular weight lower than 100,
   wherein the interfacial surface area between cetirizine and pseudoephedrine component is more than 180 mm².

The tablet dosage form can be in the form of a core tablet and coating or in the form of a bilayer tablet.

According to another aspect, there is provided a tablet dosage form comprising
a) an extended release core tablet comprising pseudoephedrine and
b) an immediate release layer comprising cetirizine over the core tablet,
   wherein the interfacial surface area between cetirizine layer and pseudoephedrine core tablet is more than 180 mm².

The process of preparation of a tablet dosage form, comprising the steps of
a) dry blending a mixture of pseudoephedrine, extended release polymer and optionally pharmaceutically acceptable inert excipients;
b) optionally granulating the blend from step a);
c) lubricating the granules from step b) or blend of step a); and compressing into a core tablet;
d) dispersing or dissolving cetirizine and optionally pharmaceutically acceptable inert excipients in a suitable solvent;
e) coating the core tablet of step c) with the dispersion or solution of step d); and
f) optionally coating the tablet of step e) with a nonfunctional coating,
   wherein the interfacial surface area between cetirizine layer and pseudoephedrine core tablet is more than 180 mm².

According to another aspect, there is provided a bilayer tablet dosage form comprising
a) an immediate release layer comprising cetirizine, and
b) an extended release layer comprising pseudoephedrine,
   wherein the interfacial surface area between cetirizine and pseudoephedrine layer is more than 180 mm².

The process of preparation of a bilayer tablet dosage form, comprising the steps of
a) dry blending a mixture of pseudoephedrine, extended release polymer and optionally pharmaceutically acceptable inert excipients;
b) optionally granulating the blend from step a);
c) lubricating the granules from step b) or the blend of step a);
d) dry blending a mixture of cetirizine and optionally pharmaceutically acceptable inert excipients;
e) optionally granulating the blend from step d);
f) lubricating the granules from step e) or the blend of step d);
g) compressing the blend or granules of step c) and f) into a bilayer tablet; and
h) optionally coating the tablet of step g) with a nonfunctional coating,
   wherein the interfacial surface area between cetirizine and pseudoephedrine layer is more than 180 mm².

The dosage forms according to any of the above aspects may further comprise alcohols having a molecular weight lower than 100.

“Cetirizine” as employed herein is intended to include not only the free compound but also any pharmaceutically acceptable salt thereof. Preferred are acid addition salts, for example, especially the dihydrochloride. “Cetirizine” is also intended to cover individual enantiomers as well as the racemate. In particular, cetirizine includes cetirizine dihydrochloride, levocetirizine dihydrochloride or efletirizine dihydrochloride.

In the present application, the term “pseudoephedrine”, used herein means pseudoephedrine itself, an individual optical isomer or a pharmaceutically acceptable salt thereof.

The term “pharmaceutically acceptable salt” as used herein with respect to pseudoephedrine can be its hydrochloride and sulfate and equivalent non-toxic salts.

The dimensions of the core tablet or the layers of bilayer tablet are chosen so that interfacial surface area between cetirizine component and pseudoephedrine component is more than 180 mm², for example, more than 200 mm², or for example, more than 220 mm².

Example of alcohols having a molecular weight lower than 100 include methanol, ethanol, isopropanol, propylene glycol, glycerin and mixtures thereof. The amount of alcohols having a molecular weight lower than 100 may range from 0.001-5% by weight of the dosage form. These may be present in the immediate release component or in the extended release component.

The term ‘extended release component’ as used herein achieves the slow release of pseudoephedrine over an extended period of time, and includes both prolonged and controlled release dosage forms.

The extended release component can further comprise extended release polymer such as polyvinylpyrrolidone, hydroxypropylcellulose, hydroxypropylmethyl cellulose, methylcellulose, vinyl acetate copolymers, polysaccharides (such as alginate, xanthan gum, etc.), polyethylene oxide, methacrylic acid copolymers, maleic anhydride/methyl vinyl ether copolymers and derivatives, ethylcellulose and polyvinylalcohols or mixtures thereof. The extended release polymer may be present at about 20-50% by weight of the total dosage form.

The tablet dosage form may further comprise pharmaceutically acceptable inert excipients selected from, for example, surfactants, binder, diluents, disintegrants, lubricants, glidants, plasticizers, stabilizers and coloring agents.

Specific examples of binders include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, propylene glycol, microcrystalline cellulose and the like.

Specific examples of diluents include lactose, calcium carbonate, calcium phosphate-dibasic, calcium phosphate-tribasic, calcium sulfate, microcrystalline cellulose, cellulose powdered and starch.

Surfactants include both non-ionic and ionic (cationic, anionic and zwitterionic) surfactants suitable for use in pharmaceutical dosage forms. These include polyethoxylated fatty acids and its derivatives, for example, polyethylene glycol 400 distearate, polyethylene glycol-20 dioleate, polyethylene glycol 4-150 mono dilaurate, polyethylene glycol-20 glyceryl stearate; alcohol-oil transesterification products, for example, polyethylene glycol-6 corn oil; polyglycerized fatty acids, for example, polyglyceryl-6 pentaoleate; propylene glycol fatty acid esters, for example, propylene glycol monocaprylate; mono and diglycerides, for example, glyceryl ricinoleate; sterol and sterol derivatives; sorbitan fatty acid esters and its derivatives, for example, polyethylene glycol-20 sorbitan monooleate, sorbitan monolaurate; polyethylene glycol alkyl ether or phenols, for example, polyethylene glycol-20 cetyl ether, polyethylene glycol-10-100 nonyl phenol; sugar esters, for example, sucrose monopalmitate; polyoxyethylene-polyoxypropylene block copolymers known as “poloxamer”; ionic surfactants, for example, sodium caproate, sodium glycocholate, soy lecithin, sodium stearyl fumarate, propylene glycol alginate, octyl sulfosuccinate disodium, palmitoyl carnitine; and the like.

Specific examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like.

Examples of coloring agents include any FDA approved colors for oral use. These may include Iron oxide, Lake of Tartrazine, Lake of Quinoline Yellow, Lake of Sunset Yellow, Aluminium Lake (blue), Lake of Erythrosine, Lack of Carmosine Ponceau, Allura Red.

Examples of suitable plasticizers include acetyl triethyl citrate, dibutyl phthalate, tributyl citrate, triethyl citrate, acetyl tributyl citrate, propylene glycol, triacetin, polyethylene glycol and diethyl phthalate.

The core tablet or bilayer tablet can be prepared by wet or dry granulation or by direct compression process.

The dosage form may optionally be coated with non-functional layers comprising film-forming polymers, if desired. There may be an intermediate seal coat present between pseudoephedrine core and cetirizine coating.

Coating may be performed by applying one or more film forming polymers, with or without other pharmaceutically inert excipients, as a solution/suspension using any conventional coating technique known in the art, such as spray coating in a conventional coating pan or fluidized bed processor; or dip coating.

The coating may be selected from amongst one or more of those suitable coating materials known in the art. For example, the coating material can be Opadry or opadry AMB (aqueous moisture barrier).

Solutions or dispersions of cetirizine or nonfunctional coating can be prepared in solvents such as, for example, dichloromethane, isopropyl alcohol, acetone, methanol, ethanol, water or mixture thereof.

The invention is further illustrated by the following examples, which is for illustrative purpose only and do not limit the scope of invention in any way.

### EXAMPLES

### Examples 1 & 2

| | | | |
|---|---|---|---|
| S. No. | Ingredients | EXAMPLE 1 | EXAMPLE 2 |

Pseudoephedrine HPMC matrix ER core

| | | | |
|---|---|---|---|
| 1 | Pseudoephedrine HCl | 120.0 | 120.0 |
| 2 | Microcrystalline cellulose | 7.9 | 7.9 |
| 3 | Glycerine | 0.1 | 0.1 |
| 4 | Isopropyl alcohol* | qs | qs |
| 5 | Hypromellose | 102.0 | 80.0 |
| | (Methocel K15M CR) | | |
| 6 | Sodium Alginate | — | 14.0 |
| | (Keltone HVCR) | | |
| 7 | Calcarb 4457 | — | 10.0 |
| 8 | Magnesium stearate | 1.5 | 1.5 |
| 9 | Talc | 1.5 | 1.5 |
| 10 | Colloidal silicon dioxide | 2.0 | 2.0 |
| | (Aerosil 200) | | |

| | | |
|---|---|---|
| Total (Core tablet weight) | 235.0 | 237.0 |

Cetirizine HCl immediate release coat

| | | | |
|---|---|---|---|
| 11 | Cetirizine HCl | 5.0 | 5.0 |
| 12 | Hypromellose (Methocel E5) | 5.0-6.0 | 5.0-6.0 |
| 13 | Polyethylene glycol 400 | 0.625-0.75 | 0.625-0.75 |
| 14 | Polyethylene glycol 6000 | 0.625-0.75 | 0.625-0.75 |
| 15 | Lactose monohydrate | 18.75-22.5 | 18.75-22.5 |
| 16 | Purified water* | qs | qs |

Opadry finish coat

| | | | |
|---|---|---|---|
| 17 | Opadry | 4.0 | 4.0 |
| 18 | Purified water* | qs | qs |

| | | |
|---|---|---|
| Total (Coated tablet weight) | 269.0-274.0 | 271.0-276.0 |

*not present in the final formulation as these are lost manufacturing.

| | | | |
|---|---|---|---|
| Average | | | |
| tablet | Thickness | Diameter | Surface area |
| parameters | (mm) | (mm) | (mm²) |

Core tablet

| | | | |
|---|---|---|---|
| EXAMPLE 1 | 4.20 | 8.50 | ~225.7 |
| EXAMPLE 2 | 4.05 | 8.50 | ~221.7 |

### Process

1. Microcrystalline cellulose and Pseudoephedrine HCl were sifted through sieve and mixed together in rapid mixer granulator (RMG).
2. Propylene glycol/glycerine was dissolved in Isopropyl alcohol and added to blend of step 1 RMG.
3. The wet mass of step 2 was dried in fluid bed dryer.
4. Dried mass of step 3 was sifted and to obtain granules.
5. Hypromellose, sodium alginate and calcium carbonate were sifted through sieve and mixed with granules,
6. Talc and Colloidal silicon dioxide (previously sifted through BSS #30) were mixed with granules of step 5.
7. Blend of step 6 were lubricated with Magnesium stearate.
8. The blend of step 7 was compressed into tablets using 8.5 mm standard concave, plain, beveled edge punches.
9. Cetirizine, hypromellose, Polyethylene glycol 400, Polyethylene glycol 6000 and Lactose monohydrate were added in purified water.
10. Tablets of Pseudoephedrine HCl from step 8 were coated with dispersion of step 9.
11. Tablets of step 10 were coated with aqueous dispersion of opadry.

Table 1 provides comparative dissolution data for the marketed Zyrtec® D and the tablet dosage form of Example 1 & 2. The testing was performed using type 1 USP dissolution apparatus, operating at 37° C. with a paddle rotating speed of 100 rpm. The tablets were tested in 900 ml of pH 1.2 hydrochloric acid for first 1 hr, followed by 900 ml of pH 6.8 phosphate buffer.

TABLE 1: Comparative dissolution data for the marketed Zyrtec® D and the tablet dosage form of Example 1 & 2.

TABLE 1
Comparative dissolution data for the marketed Zyrtec ® D
and the tablet dosage form of Example 1 & 2.

| Time | | Zyrtec ® D | Composition 1 | Composition 2 |
|---|---|---|---|---|
| | | Cumulative percentage (%) release of Pseudoephedrine HCl | | |
| 5 | min | 9 | 2 | 3 |
| 15 | min | 18 | 17 | 18 |
| 30 | min | 27 | 27 | 28 |
| 60 | min | 41 | 39 | 41 |
| 2 | hr | 64 | 63 | 67 |
| 4 | hr | 85 | 90 | 91 |
| 6 | hr | 99 | 101 | 102 |
| 8 | hr | 101 | 104 | 105 |
| 10 | hr | 103 | 105 | 106 |
| | | Cumulative percentage (%) release of Cetirizine HCl | | |
| 5 | min | 85 | 67 | 60 |
| 15 | min | 97 | 99 | 89 |
| 30 | min | 101 | 103 | 92 |
| 60 | min | 103 | 103 | 92 |

Zyrtec® D Composition 1 Composition 2 Time Cumulative percentage (%) release of Pseudoephedrine HCl

5 min 9 2 3
18 17 18
27 27 28
41 39 41
64 63 67
4 hr 85 90 91
6 hr 99 101 102
101 104 105
10 hr 103 105 106

Cumulative percentage (%) release of Cetirizine HCl

85 67 60
97 99 89
101 103 92
103 103 92

We claim:

1. A tablet dosage form comprising
a) an immediate release component comprising cetirizine or an optically active isomer thereof or pharmaceutically acceptable salt thereof and optionally pharmaceutically acceptable inert excipients; and
b) an extended release component comprising pseudoephedrine or pharmaceutically acceptable salts thereof, an extended release polymer and optionally pharmaceutically acceptable inert excipients,
wherein the interfacial surface area between cetirizine and pseudoephedrine component is more than 180 mm².

2. The tablet dosage form according to claim 1 wherein the interfacial surface area between cetirizine and pseudoephedrine component is more than 200 mm².

3. The tablet dosage form according to claim 1, wherein the dosage form further comprises alcohols having a molecular weight lower than 100 selected from methanol, ethanol, isopropanol, propylene glycol, glycerin and mixtures thereof.

4. The tablet dosage form according to claim 1, wherein the extended release polymer is selected from polyvinylpyrrolidone, hydroxypropylcellulose, hydroxypropyl methylcellulose, methylcellulose, vinyl acetate copolymers, sodium alginate, xanthan gum, polyethylene oxide, methacrylic acid copolymers, maleic anhydride/methyl vinyl ether copolymers and derivatives, ethylcellulose and polyvinylalcohols or mixtures thereof.

5. The tablet dosage form according to claim 1, wherein the extended release polymer is present in the concentration of about 20-50% by weight of the dosage form.

6. The tablet dosage according to claim 1, wherein the pharmaceutically acceptable inert excipients are selected from surfactants, binder, diluents, disintegrants, lubricants, glidants, plasticizers, stabilizers and coloring agents.

7. The tablet dosage form according to claim 1, wherein the extended release component is in the form of a core tablet and the immediate release component is present as a coating over the core tablet, and the process comprises:
a) dry blending a mixture of pseudoephedrine, extended release polymer and optionally pharmaceutically acceptable inert excipients;
b) optionally granulating the blend from step a);
c) lubricating the granules from step b) or blend of step a); and compressing into core tablet;
d) dispersing or dissolving cetirizine and optionally pharmaceutically acceptable inert excipients in a suitable solvent;
e) coating the core tablet of step c) with dispersion or solution of step d); and
f) optionally coating the tablet of step e) with a nonfunctional coating.

8. The tablet dosage form according to claim 1, wherein the dosage form is a bilayer tablet and the immediate release and extended release components are in two separate layers and the process comprises:
a) dry blending a mixture of pseudoephedrine, extended release polymer and optionally pharmaceutically acceptable inert excipients;
b) optionally granulating the blend from step a),
c) lubricating the granules from step b) or the blend of step a);
d) dry blending a mixture of cetirizine and optionally pharmaceutically acceptable inert excipients;
e) optionally granulating the blend from step d);
f) lubricating the granules from step e) or the blend of step d);
g) compressing the blend or granules of step c) and f) into a bilayer tablet; and
h) optionally coating the tablet of step g) with a nonfunctional coating.

9. The process of preparation of tablet dosage form according to claim 7 or 8 wherein granulation is by wet or dry granulation.

10. The process of preparation of tablet dosage form according to claim 9 wherein in wet granulation the granulation liquid comprises low molecular weight alcohol.
